(19) United States
(12) Patent Application Publication
(10) Pub. No.: US 2019/0071708 A1
(43) Pub. Date: Mar. 7, 2019

(54) COLLECTION BROTHS FOR MICROORGANISMS

(71) Applicant: N. Robert Ward, Bothell, WA (US)

(72) Inventor: N. Robert Ward, Bothell, WA (US)

(21) Appl. No.: 16/179,682

(22) Filed: Nov. 2, 2018

Related U.S. Application Data
(63) Continuation of application No. 13/735,798, filed on Jan. 7, 2013.
(60) Provisional application No. 61/583,438, filed on Jan. 5, 2012.

(51) Int. Cl. C12Q 1/04

(52) U.S. Cl. CPC C12Q 1/045

(57) ABSTRACT

A collection broth used for recovering microorganisms on a surface is provided. The collection broth includes a buffering system and a neutralization system. All components of the collection broth are both food-grade and hypoallergenic.

### CROSS-REFERENCE TO RELATED APPLICATION

This application claims the benefit of U.S. Application No. 61/583,438, filed Jan. 5, 2012, the disclosure of which is hereby incorporated by reference in its entirety.

### BACKGROUND

The sampling of surfaces in food production areas typically involves the use of sponges or fiber-tipped swabs that are hydrated with a collection broth or solution (also referred to in the technical literature as “transport,” “rinse” or “wetting” broth or solution). Collection broths play a critical role in the success of a surface sampling program. In the U.S., commonly used collection broths are letheen broth, neutralizing buffer, and D/E neutralizing broth.

A collection broth has two primary purposes. The first is to neutralize sanitizers that may be present on the surface that is being sampled. If a sanitizer is picked up by the sponge or swab during sample collection and is not neutralized, false negative results could occur as the collected microorganisms die from exposure to the sanitizer before the sample is processed in the laboratory.

The second purpose of the collection broth is to maintain the viability of the microorganisms after a sample is collected and until the sample is processed by the laboratory. It should be expected that the organisms that are collected on the sponge or swab are stressed due to prior exposure to a sanitizer or other injurious environmental conditions, such as drying, extremes in pH, or high or freezing temperature. In order to maintain the viability of these collected organisms, it is critical that the microorganisms be placed into a favorable and stabilizing environment that is carefully controlled for isotonicity and pH.

Sometimes the collection solution is formulated with growth-promoting nutrients. Some investigators have found that low nutrient levels are helpful in the resuscitation of injured bacteria. The presence of nutrients could complicate quantitative testing if the sample is not maintained at growth restrictive (typically 2-8° C.) temperatures after the sample is collected and there is growth of the collected microorganisms before the sample is processed in the laboratory.

The constituents of the collection broth may also aid in the collection of the sample by helping to loosen and disrupt biofilms that have formed on a surface. For example, Tween 80 (polysorbate 80), which is present in letheen and D/E neutralizing broths, may aid in release of organisms from a surface.

In addition to the important roles that the collection broths play in sample collection and cell preservation, the ideal collection broth should not interfere with the laboratory test(s) performed on the swab or sponge. However, this is not always the case. For example, sodium thiosulfate, bisulfite and citrate are reported to interfere with 3M Petrifilm. Sodium thiosulfate is present in neutralizing buffer and sodium thiosulfate and sodium bisulfite are present in D/E neutralizing broth. For many users of 3M Petrifilm, the default collection broth is letheen broth because it does not contain any of the abovementioned interfering compounds.

Developers of new generation molecular detection tests (for example, the 3M Molecular Detection Assay Listeria) have encountered interference problems with neutralizing buffer. When interference is seen, the recommendation by the companies supporting the diagnostic product is to use an alternative collection broth (frequently D/E neutralizing broth is designated) or to dilute the neutralizing buffer sufficiently with enrichment broth to eliminate the interference.

Another consideration for the selection of a collection broth relates to the appropriateness for use in a manufacturing facility. Here are some examples:

Some collection broths may contain allergenic materials that could be left on a food contact surface after sampling. Letheen broth and D/E neutralizing broth both contain lecithin, which is typically derived from soy or egg sources. D/E neutralizing broth also contains casein, which is a milk protein. While it seems unlikely that the very low concentrations of allergenic materials in a collection broth would represent a public health hazard if these materials came into contact with food, food producers may prefer not to use letheen or D/E neutralizing broth in their facility because of allergen concerns or declarations that they make to their customers about the absence of allergenic materials in their production facility.

Additionally, some pharmaceutical and biotechnology manufacturers are concerned with the use of collection broths containing animal-derived materials, because of the possibility that these animal derived materials may harbor prions responsible for Bovine Spongiform Encephalopathy (BSE) and other Transmissable Spongiform Encephalopathies (TSE). Both D/E neutralizing broth and letheen broth contain animal-derived peptones.

Finally, food manufacturing facilities that follow Kosher or Halal practices may be concerned with the use of collection broths that contain components in conflict with their traditions. Jewish dietary laws specify the separation of dairy and meat. D/E neutralizing broth, which contains casein (dairy-based) peptone, would likely be prohibited in facilities handling beef products. Halal laws forbid Muslims from eating any pork or pork-derived products. Some peptones are manufactured using porcine-derived enzymes. Consequently, even if the peptones are beef or vegetable based, they could contain some pork materials. Letheen broth contains Proteose Peptone No. 3 and D/E neutralizing broth includes a pancreatic digest of casein. For a manufacturer producing Halal certified foods, it would be important to confirm that the peptones in their collection broth do not include porcine materials.

Table 1 below shows the formulas and characteristics for the three commonly used collection broths by food industry for environmental sampling.

TABLE 1
Commonly used collection broths for environmental sampling.

| | | | | | |
|---|---|---|---|---|---|
| | | | | | Possible |
| | | | Listed in | Identified as | Allergen |
| | | | EAFUS* as | Incompatible | Associated with |
| | | Purpose in the | Acceptable in | with | Ingredient in the |
| Ingredients | Grams/Liter | Medium | Food? | 3M Petrifilm? | Solution? |

Neutralizing Buffer

| | | | | | |
|---|---|---|---|---|---|
| Monopotassium | 42.5 mg | Buffering | Yes | No | No |
| Phosphate | | | | | |
| Sodium Thiosulfate | 0.16 | Neutralizes iodine | Yes | Yes | No |
| | | and chlorine | | | |
| | | compounds | | | |
| Aryl Sulfonate | 5 | Neutralizes | No | No | No |
| Complex | | quaternary | | | |
| | | ammonium | | | |
| | | compounds | | | |

Letheen Broth

| | | | | | |
|---|---|---|---|---|---|
| Beef Extract | 5.0 | Nutrients for | Beef extract is | No | No |
| | | Growth of | not specifically | | |
| | | Microorganisms | listed but meat | | |
| | | | extract has | | |
| | | | historically | | |
| | | | been used in | | |
| | | | food products | | |
| Proteose Peptone | 10.0 | | Hydrolyzed | No | No |
| No. 3 | | | animal protein | | |
| | | | is listed | | |
| Polysorbate 80 | 5.0 | Neutralizes phenols, | Yes | No | No |
| | | hexachlorophene, | | | |
| | | formalin | | | |
| Lecithin | 0.7 | Neutralizes | Yes | No | Yes, if source is |
| | | quaternary | | | soy or egg |
| | | ammonium | | | |
| | | compounds; lecithin | | | |
| | | with polysorbate 80, | | | |
| | | neutralizes ethanol | | | |
| Sodium Chloride | 5.0 | Maintains osmotic | Yes | No | No |
| | | balance | | | |

D/E Neutralizing Broth

| | | | | | |
|---|---|---|---|---|---|
| Pancreatic Digest of | 5 | Nutrients for | Casein and | No | Yes, casein is a |
| Casein | | Growth of | hydrolyzed | | milk based |
| | | Microorganisms | milk protein are | | protein |
| | | | listed | | |
| Yeast Extract | 2.5 | | Baker's Yeast | No | No |
| | | | Extract is listed | | |
| Dextrose (glucose) | 10 | | Yes | No | No |
| Sodium | 1 | Neutralizes | No | No | No |
| Thioglycollate | | mercurials | | | |
| Sodium Thiosulfate | 6 | Neutralizes iodine | Yes | Yes | No |
| | | and chlorine | | | |
| Sodium Bisulfite | 2.5 | Neutralizes | Yes | Yes | No |
| | | formaldehyde and | | | |
| | | gluteraldehyde | | | |
| Polysorbate 80 | 5 | Neutralizes phenols, | Yes | No | No |
| | | hexachlorophene, | | | |
| | | formalin | | | |
| Lecithin | 7 | Neutralizes | Yes | No | Yes, if source is |
| | | quaternary | | | soy or egg |
| | | ammonium | | | |
| | | compounds; lecithin | | | |
| | | with polysorbate 80, | | | |
| | | neutralizes ethanol | | | |
| Bromcresol Purple | 0.02 | pH indicator for | No | No | No |
| | | acid production | | | |
| | | from microbial | | | |
| | | glucose | | | |
| | | fermentation | | | |

*Everything Added to Food in the United States

One of the challenges when developing an environmental sampling program is determining whether the collection broth used with the sampling device has sufficient capacity to neutralize the sanitizer(s) employed in all situations within a production facility. For example, a sanitizer used in the facility might be employed at a low concentration on food contact surfaces, but at far higher concentrations in locations away for the food production line (for example, an application of dry quat crystals to a zone 3 floor). Table 2 identifies the wide range of quat sanitizer concentrations that may be encountered within an aquaculture facility.

TABLE 2
Range of quat sanitizer concentrations that may
be encountered in a food production facility.

| Walls and ceilings for mold | 2,000 to 5,000 | ppm |
|---|---|---|
| Equipment sanitizing | 200 | ppm |
| Floors and drains | 800 | ppm |
| Floor mats | 1,800 | ppm |
| Foot baths | 2,400 | ppm |

While there is general information available on types of sanitizers neutralized by collection broth (as shown in Table 1), there is little to no information in the technical literature on the amount of sanitizer that each can neutralize. Furthermore, there is need for information on the ability of collection broths to neutralize newer generation sanitizers, such as peroxyacetic acid-based or fatty acid-based sanitizers. This information is generally lacking.

The second challenge in deciding on the best collection broth for a specific environment is to verify that the collection broth has the ability to maintain the viability of the microorganisms until sample processing, typically for 1 to 2 days, but in some cases up to 5 days. Microorganisms that have experienced significant injury may not remain viable during the period between collection and detection if the collection broth is not properly formulated.

Next, it is important to verify that the collection broth is appropriate for all of the laboratory tests that will be performed on the environmental sample. As mentioned earlier, certain pathogen detection tests are incompatible with neutralizing buffer. 3M Petrifilm should not be used with collection broths containing sodium thiosulfate, bisulfite and citrate.

Finally, a practical consideration is the temperature storage requirements for the collection broth. Neutralizing buffer shows good stability over a temperature range of 2 to 30° C. Manufacturers of sampling devices with letheen broth typically recommend refrigerated storage (2-8° C.). D/E neutralizing broth shows considerable pH instability and should be refrigerated (2 to 8° C.) during storage. This pH degradation results in a change in color of the broth from a typical purple color (with pH above 7.2), to a reddish color at pH 6.6 to 6.8 and a yellow color below pH 6.2. (Note: the acceptable pH range for D/E neutralizing broth is 7.4 to 7.8. D/E neutralizing broth at this pH is purple due to the presence of the pH indicator brom cresol purple.)

In view of the state of the art, improved collection broths are needed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a collection broth used for recovering microorganisms on a surface is provided. In one embodiment, the broth includes a buffering system and a neutralization system, wherein all components of the collection broth are both food-grade components and hypoallergenic components.

In another aspect, a collection broth is provided. In one embodiment, the collection broth includes:

dibasic sodium phosphate;

monobasic potassium phosphate;

sodium pyruvate;

polysorbate 80; and

lecithin derived from sunflower.

### DETAILED DESCRIPTION

In one aspect, a collection broth used for recovering microorganisms on a surface is provided. In one embodiment, the broth includes a buffering system and a neutralization system, wherein all components of the collection broth are both food-grade components and hypoallergenic components.

An exemplary version of the disclosed collection broth is referred to herein as “HiCap” (for High Capacity) neutralizing broth. HiCap is an alternative to neutralizing buffer, letheen broth and D/E neutralizing broth. HiCap neutralizing broth was formulated to meet the following specifications:

a. Has high neutralizing capacity for all sanitizers commonly used by food industry (including quaternary ammonium, peroxide/peroxyacetic, chlorine and chlorine compounds, phenolics, alcohols, and acid sanitizers);

b. Has sufficient buffering capacity to handle low-pH (high-acid) sanitizers;

c. Only includes components that are found on U.S. Food and Drug Administration's EAFUS (Everything Added to Food in the United States) list, as materials acceptable for inclusion in food;

d. Only includes components that are considered to be non-allergenic;

e. Does not contain agents (citrate, bisulfite and thiosulfate) that are reported to interfere with 3M Petrifilm;

f. Includes vegetable peptones (not digested with animal-derived enzymes) and yeast extract, instead of meat peptones or meat extracts;

g. Does not interfere with established immunoassays or newer generation molecular diagnostic tests with genetic amplification; and

h. Can be stored at a temperature range of 2 to 30° C.

The provided collection broth uses only non-toxic and non-allergenic components and has the ability to neutralize oxidizing sanitizers such as chlorine, hypochlorite, chlorine dioxide, hydrogen peroxide and peracetic acid sanitizers without the use of components such as sodium thiosulfate and sodium bisulfite. This collection broth is safe to be used in an industrial setting where products are being produced for human consumption or human contact. Moreover, this collection broth does not contain substances that are incompatible with the Petrifilm™ cold-gelation technology.

The provided collection broth only contains “food-grade” components. These are components that are identified in one of U.S. Food and Drug Administration (U.S. FDA) lists for food acceptable materials or by other reputable governmental agencies as appropriate for addition to food and are considered to be non-allergenic. In one embodiment, the food-grade components are listed as generally recognized as safe (GRAS) direct food additives in section 21 of the U.S. Code of Federal Regulations, or are included on the U.S. Food and Drug Administration's list of everything added to food in the United States (EAFUS).

The use of the provided collection broth minimizes concerns that unintentional introduction into food, pharmaceutical, or cosmetic products would cause public health or consumer acceptance issues.

Additionally, the collection broth also permits the use of a commonly used Petrifilm™ product for microbiological testing. As noted above, components of known collection products can be incompatible with the industry-standard Petrifilm™ product. Therefore, in one embodiment, no components of the collection broth are identified as incompatible with 3M™ Petrifilm™.

The collection broth only contains hypoallergenic components. Hypoallergenic ingredients in general are well known to those of skill in the art. For example, the University of Nebraska-Lincoln's Food Allergy Research and Resource Program maintains information about allergenic foods, and the Informall database of allergenic ingredients is maintained by the Institute of Food Research in the United Kingdom.

In one embodiment, the hypoallergenic components do not include components derived from any of milk, eggs, fish, crustacean and molluscan shellfish, tree nuts, peanuts, wheat, soybean, buckwheat, celery, lupin, mustard, and sesame.

The collection broth includes a buffering system and a neutralization system. The collection broth is typically an aqueous liquid mixture.

The buffering system of the collection broth acts to maintain the pH of the collected sample when in contact with the collection broth. In one embodiment, the buffering system results in a pH in the range of 6 to 8 when the collection broth is mixed with high-acid sanitizers, including peracetic acid type sanitizers employed at typical use concentrations. Other acids that the buffering system may come into contact with during collection include, for example, acetic acid; nitric acid; phosphoric acid; 1-octanesulfonic acid, sodium salt; nonanoic acid, and capric acid.

In one embodiment, the buffering system comprises anhydrous potassium phosphate dibasic and anhydrous potassium phosphate monobasic. In one embodiment, the buffering system comprises anhydrous potassium phosphate dibasic at a concentration from about 1 grams/liter to about 15 grams/liter and anhydrous potassium phosphate monobasic at a concentration from about 1 gram/liter to about 15 grams/liter.

The neutralization system neutralizes the active ingredients in sanitizers in order to prevent harm to microorganisms collected with the collection broth. Commonly used sanitizers include quaternary ammonium, halogen, and peracetic acid sanitizers.

In one embodiment, the neutralization system comprises pyruvate. The provided collection broth uses pyruvate for the triple purposes of: (i) removing free radicals formed during gamma sterilization of a collection broth prior to sample collection, (ii) providing the collection broth with the ability to neutralize oxidizing sanitizers such as chlorine and peroxide-based sanitizers on a surface, without the use of Petrifilm™ incompatible components such as sodium thiosulfate, sodium thioglycollate or sodium bisulfate; and (iii) enhancing the recovery of bacteria collected from a surface that may be injured. In one embodiment, the pyruvate is present at a concentration from about 1 grams/liter to about 10 grams/liter. In one embodiment, the pyruvate is selected from the group consisting of pyruvic acid, sodium pyruvate, ethyl pyruvate, hexenyl pyruvate, isoamyl pyruvate, and propyl pyruvate.

In one embodiment, the neutralization system comprises polysorbate 80. Polysorbate 80 is useful to neutralize phenols, hexachlorophene, and formalin. In one embodiment, polysorbate 80 is present at a concentration from about 5 grams/liter to about 20 grams/liter.

In one embodiment, the neutralization system comprises lecithin. Lecithin neutralizes quaternary ammonium sanitizers (the combination of lecithin and polysorbate 80 neutralize alcohol sanitizers). In one embodiment, the lecithin is present at a concentration from about 5 grams/liter to about 10 grams/liter. In certain embodiments, lecithin derived from sunflowers (considered to be a non-allergenic source of lecithin) is used. Soy and egg are the more typical sources of lecithin. In other embodiments, canola lecithin is used.

In one embodiment, the neutralization system comprises pyruvate, polysorbate 80, and lecithin.

A nutrient component can be included in the collection broth in order to aid the growth of collected microorganisms. In one embodiment, the collection broth further comprises a nutrient component, wherein the nutrient component is not derived from an animal, is food-grade, and is hypoallergenic. Representative nutrient components include yeast extract and vegetable-based peptones. In one embodiment, the nutrient component is selected from the group consisting of carbohydrates (e.g., glucose), proteins, and peptones. Peptones are enzymatic or chemically digested proteins.

In one embodiment, the nutrient component comprises a yeast extract prepared using animal-free materials. In one embodiment, the yeast extract is present at a concentration from about 1 grams/liter to about 10 grams/liter.

In one embodiment, the nutrient component comprises a vegetable-based peptone. In one embodiment, the vegetable-based peptone is present at a concentration from about 1 grams/liter to about 20 grams/liter.

In one embodiment, no components of the collection broth are identified as incompatible with 3M™ Petrifilm™. Many commonly used components of collection broths are not compatible with Petrifilm, including sodium thiosulfate, bisulfite, and citrate, as reported by the American Public Health Association in Standard Methods for the Examination of Dairy Products (2004), page 174.

Set forth below in Example 6 is a study that evaluated the compatibility of HiCap with Petrifilm, as well as HiCap with various food pathogen detection tests. In the study, HiCap was found to be compatible with Petrifilm during all tests. The results also show compatibility between HiCap and two immunoassays (VIDAS and Romer) and two molecular detection technologies (BAX and Neogen ANSR).

In another aspect, a collection broth is provided. In one embodiment, the collection broth includes:
dibasic sodium phosphate;
monobasic potassium phosphate;
sodium pyruvate;
polysorbate 80; and
lecithin derived from sunflower.

In one embodiment, the collection broth further comprises vegetable-based peptones.

In one embodiment, the collection broth further comprises yeast extract.

In one embodiment, the collection broth is an aqueous preparation that consists essentially of:
dibasic sodium phosphate;
monobasic potassium phosphate;
sodium pyruvate;
polysorbate 80; and
lecithin derived from sunflower.

In one embodiment, the collection broth is an aqueous preparation that consists essentially of:
dibasic sodium phosphate;
monobasic potassium phosphate;
sodium pyruvate;
polysorbate 80;
lecithin derived from sunflower;
and a nutrient component.

In one embodiment, the nutrient component is vegetable-based peptones and/or yeast extract.

In another aspect, a method of sampling a surface for microorganisms using the disclosed collection broth is provided. In one embodiment, the method includes contacting the surface with an absorbent material containing a collection broth according to disclosed embodiments. Typical microorganism collection methods can be combined with the disclosed collection broth.

In one embodiment, the absorbent material is a sponge or swab. For example, World Bioproducts LLC (Bothell, Wash.) sells a product called EZ-Reach Sponge Sampler (with the Item No. EZ-10HC-PUR) that contains a polyurethane sponge with 10 mL of HiCap Neutralizing Broth that can be used with the disclosed methods.

In one embodiment, the method further comprises testing the transport broth for microorganisms. Testing methods are known to those of skill in the art. Representative testing methods include quantitative tests where an aliquot of the collection broth (after the surface sample is collected using a sponge or swab) is inoculated (i) onto the surface of an agar-based culture medium in a Petri dish (called a spread plate method) or (ii) into an empty Petri dish where a warm, molten agar-based culture medium is added and mixed with the inoculum and allowed to solidify (called a pour plate method). Alternatively, 1 ml aliquot of the collection broth could be added to a 3M Petrifilm™ plate. In all cases, the Petri dish or the Petrifilm is incubated, typically for 24 to 48 hours, at 35° C. Individual colonies that have developed on or in the agar-based culture medium or within the gel of the Petrifilm are counted to provide quantitative results. Representative testing methods also include qualitative tests where the collection broth is tested for the presence of a particular type of organism, usually a bacterial pathogen such as Salmonella, Listeria or Escherichia coli. A common approach is to add the sponge with the collected microorganisms into a culturing broth (often called an enrichment broth). This broth is typically incubated for 6 to 48 hours at 30 to 35° C. to grow the organism of interest. This broth may be tested for the presence of the microorganism of interest using an immunoassay test, such as bioMerieux's VIDAS test, or a molecular detection test, such as Dupont's BAX test.

The following examples are included for the purpose of illustrating, not limiting, the disclosed embodiments.

### EXAMPLES

### Example 1. Exemplary Collection Broth

A collection broth containing the components listed in Table 3 was tested for its ability to neutralize a quaternary ammonium sanitizer and a peroxide sanitizer. This collection broth was found to be capable of neutralizing at least 200 mg/L (ppm) of a quaternary ammonium sanitizer (Virex, JohnsonDiversey, Sturtevant, Wis.) and peracetic acid sanitizer (Minncare Cold Sterilant, Minntech Corporation, Minneapolis, Minn.). This collection broth contains none of the components identified as incompatible with Petrifilm (sodium thiosulfate and sodium bisulfite) but maintains the capacity to neutralize commonly used sanitizers. Also, all components are acceptable for use in food, as identified in EAFUS list of the U.S. FDA.

TABLE 3
Composition of exemplary collection broth.

| | | | On Governmental | | |
|---|---|---|---|---|---|
| | | | Lists (GRAS, | Identified as | Known Allergenic |
| | | | EAFUS) of | Incompatible | Properties |
| | | Purpose in the | Substances Acceptable | with 3M | Associated with |
| | Grams/Liter | Medium | in Food? | Petrifilm? | the Material? |
| Vegetable Based | 5 | Nutrients for Growth | Hydrolyzed Vegetable | No | No |
| Peptones | | of Microorganisms | Protein is listed | | |
| Yeast Extract | 1 | | Baker's Yeast Extract | No | No |
| | | | is Listed | | |
| Sodium Phosphate, | 7.75 | Maintain neutral pH | Yes | No | No |
| Dibasic | | of collection broth; | | | |
| Potassium Phosphate, | 2.25 | neutralize sanitizer | Yes | No | No |
| Monobasic | | with acidic or basic | | | |
| | | properties. | | | |
| Sodium Pyruvate | 10 | Neutralizes oxidizing | Yes | No | No |
| | | sanitizers, such as | | | |
| | | chlorine and | | | |
| | | peroxides | | | |
| Polysorbate 80 | 5 | Neutralizes phenols, | Yes | No | No |
| | | hexachlorophene, | | | |
| | | formalin | | | |
| Lecithin, from | 7 | Neutralizes | Yes | No | No |
| Sunflower | | quaternary | | | |
| | | ammonium sanitizers | | | |
| | | (the combination of | | | |
| | | lecithin and | | | |
| | | polysorbate 80 | | | |
| | | neutralize alcohol | | | |
| | | sanitizers) | | | |

### Example 2. Simplified Exemplary Collection Broth (HiCap)

This collection broth is identical to the one in Example 1 without any nutrients for microbial growth. A collection broth without nutrients is preferable in certain applications where it would be inappropriate to apply nutrients to the surface because of concern that these nutrients might support microbial growth subsequent to sample collection.

TABLE 4
Composition of exemplary collection broth.

| | | | On Governmental | | |
|---|---|---|---|---|---|
| | | | Lists (GRAS, | Identified as | Known Allergenic |
| | | | EAFUS) of | Incompatible | Properties |
| | | Purpose in the | Substances Acceptable | with 3M | Associated with |
| | Grams/Liter | Medium | in Food? | Petrifilm? | the Material? |
| Sodium Phosphate, | 7.75 | Maintain neutral pH | Yes | No | No |
| Dibasic | | of collection broth; | | | |
| Potassium Phosphate, | 2.25 | neutralize sanitizer | Yes | No | No |
| Monobasic | | with acidic or basic | | | |
| | | properties | | | |
| Sodium Pyruvate | 10 | Neutralizes oxidizing | Yes | No | No |
| | | sanitizers, such as | | | |
| | | chlorine and | | | |
| | | peroxides | | | |
| Polysorbate 80 | 5 | Neutralizes phenols, | Yes | No | No |
| | | hexachlorophene, | | | |
| | | formalin | | | |
| Lecithin, from | 7 | Neutralizes | Yes | No | No |
| Sunflower | | quaternary | | | |
| | | ammonium sanitizers | | | |
| | | (the combination of | | | |
| | | lecithin and | | | |
| | | polysorbate 80 | | | |
| | | neutralize alcohol | | | |
| | | sanitizers) | | | |

### Example 3: 48 Hour Survival Experiments

These experiments were designed to assess whether HiCap neutralizing broth can maintain the viability of cells over a 48 hour period at refrigerated temperatures.

Method: These experiments were conducted by squeezing HiCap neutralizing broth from sterile EZ Reach™ Sponge Sampling units (with polyurethane sponges) and dispensing into sterile polypropylene vials. Low levels of Escherichia coli, Staphylococcus aureus, Salmonella enterica serotype Typhimurium, and Listeria monocytogenes were inoculated into containers with the HiCap neutralizing broth. Counts, using a pour plate method with Standard Methods Agar (SMA), were made at time of inoculation (Time=0) and after 24 hours (Time=24 hr) and 48 hours (Time=48 hr) of holding at refrigerated temperatures (4-8° C.).

Results: Table 5 below shows that these test organisms maintained their viability over 48 hours of refrigerated storage.

TABLE 5
Survival of test organisms in HiCap neutralizing broth
held at refrigerated temperatures for 48 hours.

| | Counts in HiCap neutralizing broth held for 24 and | | |
|---|---|---|---|
| | 48 hours at 4-8° C. (expressed as log mean colony | | |
| | forming units (CFU) per milliliter) | | |
| Test Organisms: | Time = 0 Hr | Time = 24 Hr | Time = 48 Hr |
| Escherichia coli | 2.6 | 2.6 | 2.6 |
| Staphylococcus aureus | 2.5 | 2.6 | 2.6 |
| Salmonella enterica serotype | 2.7 | 2.6 | 2.7 |
| Typhimurium | | | |
| Listeria monocytogenes | 2.6 | 2.6 | 2.6 |

### Example 4: pH Neutralization Experiment

These experiments were designed to test whether neutralizing buffer, letheen broth, D/E neutralizing broth and HiCap neutralizing broth are able to establish a neutral pH when mixed with sanitizer.

Some sanitizers have very low pH. For example, two sanitizers from Ecolab Inc. (Vortexx™ and Mandate™ Plus) are defined as acid sanitizers. A 1% solution of Vortexx has a pH of 2.5 and whereas an undiluted preparation of Mandate Plus has a pH of 1. Tsunami® 200, also from Ecolab, is used for produce wash and has a pH of 2.5 (1% solution).

On the alkaline side of the pH spectrum, Whisper™ V, which is a 5th generation quat sanitizer from Ecolab, has a pH of 7.7 (100% solution). XY-12® is a sodium hypochlorite sanitizer with pH of 8.3 (25% solution).

It is important that a neutralizing solution has sufficient buffering capacity to neutralize low and high pH sanitizers. Microorganisms when collected during sampling of a surface may be sublethally injured due to exposure to a sanitizer, starvation, freezing, heating, high or low pH, osmotic shock, and desiccation. The viability of these injured cells may be better maintained in a collection broth that is sufficiently buffered to overcome an acid sanitizer and bring pH into an optimal range of 6-8.

This experiment was conducted to compare the pH adjusting capacity of neutralizing buffer, letheen broth, D/E neutralizing broth and HiCap neutralizing broth when mixed with different types of sanitizers.

Method: Sterile solutions of neutralizing buffer, letheen broth, D/E neutralizing broth and HiCap neutralizing broth were dispensed in sterile polypropylene vials. Sanitizer was added to each tube to establish a final dilution of 1:400. The pH of each solution after addition of the sanitizer was determined.

Results: Table 6 shows results of pH measurements after mixing four collection broths with five different sanitizers. The data show that neutralizing buffer, letheen broth and D/E neutralizing broth have insufficient buffering capacity when mixed with acid sanitizers (Vortexx, Tsunami 200 and Mandate Plus) to adjust the pH above 6. HiCap neutralizing buffer, when mixed with any of the sanitizers tested, resulted in pH values between 6.5 and 7.2.

TABLE 6
pH of collection broths mixed with different types of sanitizers.

| | 1:400 Dilution of | 1:400 Dilution of | | | |
|---|---|---|---|---|---|
| | Vortexx ™* (Hydrogen | XY-12 ® | | | |
| | peroxide (6.9%); | (Sodium | | | 1:400 Dilution of |
| | Peroxyacetic acid | Hypochlorite | | 1:400 Dilution of | Mandate ™ Plus |
| | (4.4%); Octanoic Acid | 8.4%) - Use | | Tsunami ® 200 | (Nonanoic |
| | (3.3%)) - Use dilutions | dilution of 1:416 | | (Peracetic acid | (Pelargonic) Acid |
| | recommended by | for nonporous | | (13%); Hydrogen | 6.30%; Decanoic |
| | manufacturer are 1:200 | food contact | 1:400 Dilution of Whisper ™ | peroxide (1-5%); | (Capric) Acid |
| | to 1:1000 for non-food | surfaces is | V (mixed quaternary | Caprylic Acid | 1.09%) - Use |
| | contact surfaces; Use | recommended by | ammonium sanitizers) - Use | (5-20%)) - Use | dilution recommended |
| | dilutions are 1:500 to | manufacturer if no | dilutions are 1:63 to | dilution recommended | by manufacturer is |
| Neutralizing | 1:1000 for food contact | test kit is | 1:256 are recommended | by manufacturer is | 1:427 for coarse |
| Solution | surfaces | available. | by manufacturer. | 1:3167 for produce wash | spray application |
| Neutralizing Buffer | 4.3 | 7.0 | 7.5 | 3.8 | 3.6 |
| Letheen Broth | 4.7 | 6.8 | 6.8 | 4.4 | 4.6 |
| D/E | 5.3 | 7.4 | 7.5 | 4.8 | 5.6 |
| Neutralizing Broth | | | | | |
| HiCap | 6.7 | 7.2 | 7.2 | 6.5 | 6.8 |
| Neutralizing Broth | | | | | |

*Vortexx, XY-12, Whisper, Tsunami, and Mandate are trademarks of Ecolab Inc., St. Paul, MN

### Example 5: Sanitizer Neutralization Experiments

These experiments were performed to assess the neutralization capacities of letheen broth, neutralizing buffer, D/E neutralizing broth and HiCap neutralizing broth with 4 types of sanitizers commonly used by food industry. One or more sanitizers representing the following sanitizer types were included in this study.

(1) Chlorine

(2) Quaternary ammonium compounds

(3) Peroxide and peroxyacetic acid mixtures

(4) Mixed acid

Method: One milliliter aliquots of sterile solutions of neutralizing buffer, letheen broth, D/E neutralizing broth and HiCap neutralizing broth were dispensed into sterile polypropylene vials. Each sanitizer was diluted in sterile water and 0.010 ml was added to the individual tubes to give final concentrations of 1:100, 1:200, 1:400, 1:800, 1:1600 and 1:3200. Ten microliters of a diluted culture of Escherichia coli ATCC 25922 (giving a final concentration of approximately 500 to 1000 cells per ml) was added to each tube. Controls were also performed. One control is a sanitizer activity control which was performed as above, with the exception that sterile purified water was used instead of a collection broth. This control verifies that the sanitizer at the test concentration is lethal to E. coli in the absence of the collection broth. A growth control was performed as above, with the exception that no sanitizer was added to the collection broth.

Each tube was incubated at refrigerated temperatures (4-8° C.) for 60 minutes. One ml of a sterile 2× solution of tryp soy broth supplemented with 1% yeast extract was then added to the neutralization tubes. All tubes were incubated for 24+/−2 hours at 35° C. and observed for turbidity. The presence of turbidity indicated that a sanitizer was successfully neutralized by the collection broth.

Chlorine Sanitizer Neutralization Studies:

XY-12 contains 8.4% sodium hypochlorite as the active ingredient. Table 7 shows the neutralization activity profiles of D/E neutralizing broth, letheen broth, neutralizing buffer, and HiCap neutralizing broth with XY-12.

TABLE 7
Neutralization activity profiles of four collection broths with XY-12 chlorine sanitizer.

| | | Collection | Dilution of Sanitizer Used in Assay | | | | | |
|---|---|---|---|---|---|---|---|---|
| Sanitizer Type | Manufacturer | Broth | 1:100 | 1:200 | 1:400 | 1:800 | 1:1600 | 1:3200 |
| XY-12 (Sodium | Ecolabs | D/E | + | + | +* | + | + | + |
| Hypochlorite | | Neutralizing | | | | | | |
| 8.4%) - Use | | Broth | | | | | | |
| dilution is 1:833 | | Letheen | − | − | +* | + | + | + |
| for nonporous | | Broth | | | | | | |
| food contact | | Neutralizing | − | − | −* | + | + | + |
| equipment (Use | | Buffer | | | | | | |
| dilution of 1:416 | | HiCap | + | + | +* | + | + | + |
| for nonporous | | Neutralizing | | | | | | |
| food contact | | Broth | | | | | | |
| surfaces if no test | | | | | | | | |
| kit is available) | | | | | | | | |

Note 1.
*indicates the highest concentration of sanitizer (lowest use dilution) recommended by the manufacturer.
Note 2.
+ is positive for neutralization in the bioassay. Without neutralization, this concentration is lethal to the test organism.

The greatest neutralization capacity against XY-12 was seen with D/E neutralizing broth and HiCap neutralizing broth. The weakest neutralization activity was seen with neutralizing buffer, which showed neutralizing activity only when the XY-12 was diluted 1:800.

Quat Sanitizes Neutralization Studies:

Whisper V and Virex® 256, both fifth generation quat sanitizers, were analyzed in this neutralization study. Table 8 shows the neutralization activity profiles of D/E neutralizing broth, letheen broth, neutralizing buffer, and HiCap neutralizing broth with these mixed quat sanitizers.

TABLE 8
Neutralization activity profiles of four collection broths with two quat preparations.

| | | Collection | Dilution of Sanitizer Used in Assay | | | | | |
|---|---|---|---|---|---|---|---|---|
| Sanitizer Type | Manufacturer | Broth | 1:100 | 1:200 | 1:400 | 1:800 | 1:1600 | 1:3200 |
| Whisper V | Ecolabs | D/E | +* | + | + | + | + | + |
| (mixed quaternary | | Neutralizing | | | | | | |
| ammonium | | Broth | | | | | | |
| sanitizers Alkyl | | Letheen | −* | − | + | + | + | + |
| dimethyl benzyl | | Broth | | | | | | |
| ammonium | | Neutralizing | +* | + | + | + | + | + |
| chloride 3.00%; | | Buffer | | | | | | |
| Octyl decyl | | HiCap | +* | + | + | + | + | + |
| dimethyl | | Neutralizing | | | | | | |
| ammonium | | Broth | | | | | | |
| chloride 2.25%; | | | | | | | | |
| Didecyl dimethyl | | | | | | | | |
| ammonium | | | | | | | | |
| chloride (1.35%); | | | | | | | | |
| Dioctyl dimethyl | | | | | | | | |
| ammonium | | | | | | | | |
| chloride 0.9%) - | | | | | | | | |
| Use dilutions are | | | | | | | | |
| 1:63 to 1:256 | | | | | | | | |
| Virex 256 | Diversey | D/E | + | +* | + | + | + | |
| (Didecyl dimethyl | (Virex is a | Neutralizing | | | | | | |
| ammonium | registered | Broth | | | | | | |
| chloride | trademark of | Letheen | − | −* | − | + | + | |
| (8.704%); n-alkyl | Diversey, Inc., | Broth | | | | | | |
| dimethyl benzyl | Sturtevant, WI | Neutralizing | − | +* | + | + | + | |
| ammonium | | Buffer | | | | | | |
| chloride | | HiCap | + | +* | + | + | + | |
| (8.190%)) - Use | | Neutralizing | | | | | | |
| dilution is 1:256 | | Broth | | | | | | |

Note 1.
*indicates the highest concentration of sanitizer (lowest use dilution) recommended by the manufacturer.

Letheen broth showed a lower ability to neutralize both quat sanitizers when compared to the other three collection broths. With the Virex 256 preparation, D/E neutralizing broth and HiCap neutralizing broth showed a higher neutralization capability than neutralizing buffer.

Peroxide-Peroxyacetic Acid Sanitizes Neutralization Studies:

Two peroxide-based sanitizers, Vortexx and Tsunami 200, were studied in these neutralization experiments. Table 9 shows the neutralization profiles of the four collection broths with these sanitizers.

TABLE 9
Neutralization activity profiles of four collection broths with peroxide-based sanitizers

| | | Collection | Dilution of Sanitizer Used in Assay | | | | | |
|---|---|---|---|---|---|---|---|---|
| Sanitizer Type | Manufacturer | Broth | 1:100 | 1:200 | 1:400 | 1:800 | 1:1600 | 1:3200 |
| Vortexx | Ecolabs | D/E | − | +* | + | + | + | + |
| (Hydrogen | | Neutralizing | | | | | | |
| peroxide (6.9%); | | Broth | | | | | | |
| Peroxyacetic acid | | Letheen | − | −* | − | − | − | + |
| (4.4%); Octanoic | | Broth | | | | | | |
| Acid (3.3%)) - | | Neutralizing | − | −* | − | − | + | + |
| Use dilutions are | | Buffer | | | | | | |
| 1:200 to 1:1000 | | HiCap | + | +* | + | + | + | + |
| for nonfood | | Neutralizing | | | | | | |
| contact surfaces; | | Broth | | | | | | |
| Use dilutions are | | | | | | | | |
| 1:500 to 1:1000 | | | | | | | | |
| for food | | | | | | | | |
| contact surfaces | | | | | | | | |
| Tsunami 200 | Ecolabs | D/E | − | + | + | + | + | + |
| (Peracetic acid | | Neutralizing | | | | | | |
| (13%); Hydrogen | | Broth | | | | | | |
| peroxide (1-5%); | | Letheen | − | − | − | − | + | + |
| Caprylic Acid | | Broth | | | | | | |
| (5-20%)) - Use | | Neutralizing | − | − | − | − | + | + |
| dilution is 1:3167 | | Buffer | | | | | | |
| | | HiCap | − | + | + | + | + | + |
| | | Neutralizing | | | | | | |
| | | Broth | | | | | | |

Note 1.
*indicates the highest concentration of sanitizer (lowest use dilution) recommended by the manufacturer.

Both letheen broth and neutralizing buffer showed little neutralization capability with the Vortexx and Tsunami 200 preparations. In contrast, both D/E neutralizing broth and HiCap neutralizing broth showed a high capacity to neutralize these peroxide-based sanitizers.

Mixed Acid Sanitizer Neutralization Studies:

Mandate Plus is an acid sanitizer with two active ingredients (nonanoic (pelargonic) acid and decanoic (capric) acid). Mandate Plus also contains acetic acid, nitric acid, phosphoric acid and octanesulfonic acid.

Table 10 shows the neutralization profiles of the four collection broths with this acid sanitizer.

TABLE 10
Neutralization activity profiles of four collection broths with Mandate Plus acid sanitizer.

| | | Collection | Dilution of Sanitizer Used in Assay | | | | | |
|---|---|---|---|---|---|---|---|---|
| Sanitizer Type | Manufacturer | Broth | 1:100 | 1:200 | 1:400 | 1:800 | 1:1600 | 1:3200 |
| Mandate Plus | Ecolabs | D/E | − | + | +* | + | + | + |
| (Nonanoic | | Neutralizing | | | | | | |
| (Pelargonic) Acid | | Broth | | | | | | |
| 6.30%; Decanoic | | Letheen | − | − | +* | + | + | + |
| (Capric) Acid | | Broth | | | | | | |
| 1.09%) - Use | | Neutralizing | − | − | −* | − | + | + |
| dilution is 1:427 | | Buffer | | | | | | |
| for coarse spray | | HiCap | − | + | +* | + | + | + |
| application | | Neutralizing | | | | | | |
| | | Broth | | | | | | |

Note 1.
*indicates the highest concentration of sanitizer (lowest use dilution) recommended by the manufacturer.

Neutralizing buffer showed very little capacity to neutralize Mandate Plus. Letheen broth was able to neutralize this acid sanitizer when diluted 1:400. The greatest neutralization capability was seen with the D/E neutralizing broth and the HiCap neutralizing broth.

### Example 6: New Generation Diagnostic Tests

HiCap Neutralizing broth was evaluated in a study conducted by Cherney Microbiological Services, Ltd (Green Bay, Wis.) to test for compatibility with two immunoassays and two molecular detection assays used by food industry to detect Salmonella and Listeria in a sample.

Method:

The following Listeria organisms were grown and mixed equally to make a suspension (“cocktail”) of approximately 30 cells/0.1 ml:

Listeria ivanovii ATCC 19119

Listeria monocytogenes ATCC 19114

Listeria innocua ATCC 33090

Listeria monocytogenes ATCC 7644

The following Salmonella genus organisms were grown and mixed equally to make a suspension (“cocktail”) of approximately 30 cells/0.1 ml:

Salmonella enterica subsp enteritidis ATCC 13076

Salmonella enterica subsp choleraesuis ATCC 10708

Salmonella enterica subsp abaetetuba ATCC 35640

Salmonella bongori ATCC 43975

Salmonella enterica subsp Newport ATCC 6962

The 0.1 ml of each cocktail was used to inoculate environmental sponges with HiCap Neutralizing Broth. Immediately prior to inoculating the sponges, the inoculum was pour plated on SPC agar which produced Listeria counts of 42 colonies/0.1 ml and Salmonella counts of 34 colonies/0.1 ml. The sponges inoculated with the Listeria spp. or Salmonella spp. cocktails were placed in the refrigerator at 4° C. for 72 hours. The sponges were then processed for each pathogen cocktail by following the enrichment and detection protocols recommended by the manufacturers of the tests. Environmental sponges with HiCap Neutralizing Broth, which were not inoculated with the microorganism cocktails, were also tested. These are identified in Table 11 below as “HiCap Only”.

TABLE 11
Neutralization activity profiles of four collection
broths with Mandate Plus acid sanitizer.

| | Detection Test Type | | | | | | | |
|---|---|---|---|---|---|---|---|---|
| | | | | | Molecular | | Molecular | |
| | Automated | | Lateral Flow | | Detection | | Detection | |
| | Immunoassay | | Immunoassay | | Assay | | Assay | |
| | Manufacturer and Test Name | | | | | | | |
| | bioMerieux | | Romer | | DuPont | | Neogen | |
| | VIDAS | | RapidChek | | BAX Q7 | | ANSR | |
| | Rep 1 | Rep 1 | Rep 2 | Rep 2 | Rep 1 | Rep 2 | Rep 1 | Rep 2 |
| HiCap + | + | + | + | + | + | + | + | + |
| Listeria spp. | | | | | | | | |
| HiCap + | + | + | + | + | + | + | + | + |
| Salmonella spp. | | | | | | | | |
| HiCap Only | − | − | − | − | − | − | − | − |
| (Listeria assay) | | | | | | | | |
| HiCap Only | − | − | − | − | − | − | − | − |
| (Salmonella assay) | | | | | | | | |

These studies confirmed compatibility of HiCap Neutralizing Broth with diagnostic tests used by food industry for testing the presence of Listeria and Salmonella pathogens in environmental samples.

### Example 7: Conclusions and Discussion

The selection of a collection broth for an environmental sampling program should include an assessment of whether the collection broth can maintain the viability of the collected microorganisms until the sample is processed in the laboratory and whether the collection broth can effectively neutralize all sanitizer(s) used in all places in the production facility. Our work indicates that HiCap neutralizing broth squeezed from EZ Reach Sponge Samplers with polyurethane sponges is able to maintain the viability of E. coli, Salmonella, S. aureus and L. monocytogenes over a 48 hour period at refrigerated temperatures.

Maintenance of viability is likely enhanced when the collected microorganisms are immediately placed into a culture environment favorable to the resuscitation of these cells. There are many factors that come into play when considering the resuscitation of injured microorganisms.

Maintaining a neutral pH may improve recovery of injured bacteria. In our studies, we found that when acid sanitizers were mixed with traditional collection broths that the pH remained low. For example, when neutralizing buffer was mixed with 3 sanitizers with high acidity (Vortexx, Tsunami 200 and Mandate Plus) that had been diluted 1:400, the pH remained below pH 4.5. Letheen broth and D/E neutralizing broth were only slightly better than neutralizing buffer for raising pH when mixed with these high acid sanitizers. HiCap neutralizing buffer was formulated with a sufficient buffering system to overcome the acidity of these low pH sanitizers.

In our studies, D/E and HiCap neutralizing broths show excellent neutralization capacities with all of the sanitizers tested. Letheen broth was found to have 25% (or less) of the neutralization capacity of the other 3 collection broths when tested with quat sanitizers. Both neutralizing buffer and letheen broth showed a poor ability to neutralize peroxide-based sanitizers. Finally, neutralizing buffer showed a reduced capacity to neutralize sodium hypochlorite (XY-12) and an acid sanitizer (Mandate Plus).

For companies that employ a rotation of sanitizers to minimize selection of resistant organisms, it is especially important to confirm that the collection broth employed in their environmental monitoring program has the ability to neutralize all of the sanitizers at all of the concentrations used in the production facility. This confirmation is only possible by knowing the amount of sanitizer likely to be present on the area to be sampled and an understanding of the neutralization capabilities of the collection device.

Here is an example to illustrate this point. Our data indicate that 1 ml of letheen broth has a neutralization limit of about 0.2 mg for Virex 256 (10 mls of letheen would have a neutralization limit of about 2 mg of Virex 256). When we have estimated the amount of Virex 256 that might be present on a non-porous surface (when diluted according to the manufacturer's recommendation, evenly spread and dried onto the non-porous surface), we find that a 100 cm² area may have up to 0.7 mg or a 1 ft² area may have up to 6.3 mg of Virex 256. What this suggests is that 10 mls of letheen broth would be adequate for sampling a smaller area (4 int or 100 cm²), but might be inadequate to fully neutralize the amount of sanitizer on a 1 ft² area. One ml of letheen broth may be inadequate for sampling an area greater than about 25 cm².

Concern for reaching a neutralization limit during sampling is substantially mitigated by employing a collection broth with sufficient capacity. For example, 1 ml of HiCap neutralizing broth has the capacity to neutralize more than 1.7 mg of Virex 256, which should provide ample neutralization capacity to sample an area of 100 cm² when the quat sanitizer is applied to the surface. Ten ml of HiCap can neutralize more than 17 mg of Virex 256. The use of a higher volume of HiCap (5 to 10 ml) may merit consideration when sampling large areas such as 1 ft².

In addition to the critical requirements that a collection broth effectively neutralize residual sanitizer and maintain the viability of the collected microorganisms, other considerations may come into play when selecting a collection broth. These include compatibility with laboratory procedures, the potential presence of allergenic components or animal derived materials in the collection broth, acceptability of the collection broth in contact with food, temperature storage requirements, and compatibility with Kosher and Halal practices.

HiCap neutralizing broth was formulated to have universal application for surface sampling with features included to help assure that an environmental monitoring program produces meaningful information for the food company.

While illustrative embodiments have been illustrated and described, it will be appreciated that various changes can be made therein without departing from the spirit and scope of the invention.

1-20. (canceled)

21. A method of collecting microorganisms from a surface, comprising:
providing a sponge or a swab comprising a collection solution comprising a pH buffering system and a sanitizer neutralization system,
wherein the pH buffering system results in a pH in the range of 6 to 8 when the collection solution is mixed with an acid sanitizer with a pH at 2.5 comprising an acid selected from peracetic acid, octanoic acid, and capric acid,
wherein the sanitizer neutralization system comprises pyruvate and sunflower lecithin,
wherein the collection broth does not include components derived from milk, eggs, fish, crustacean and molluscan shellfish, tree nuts, peanuts, wheat, soybean, buckwheat, celery, lupin, mustard, and sesame, and
wherein the collection solution does not comprise citrate, bisulfite, or thiosulfate ions, or any combination thereof;
contacting a surface with the sponge or swab comprising the collection solution to collect microorganisms on the surface;
optionally storing the sponge or swab comprising the collection solution at a temperature of 2 to 8° C.; and
testing the sponge or swab comprising the collected microorganisms quantitatively using an aliquot of the collection solution in the sponge or swab, or qualitatively by exposing the sponge or swab to a culturing medium.

22. The method of claim 21, further comprising culturing the collected microorganisms in the culturing medium.

23. The method of claim 21, wherein the pH buffering system comprises potassium phosphate dibasic or sodium phosphate dibasic, and potassium phosphate monobasic.

24. The method of claim 23, wherein the pH buffering system comprises potassium phosphate dibasic or sodium phosphate dibasic at a concentration from about 1 gram/liter to about 15 grams/liter, and potassium phosphate monobasic at a concentration from about 1 gram/liter to about 15 grams/liter.

25. The method of claim 21, wherein the pyruvate is present at a concentration from about 1 gram/liter to about 10 grams/liter.

26. The method of claim 21, wherein the pyruvate is selected from the group consisting of pyruvic acid, sodium pyruvate, ethyl pyruvate, hexenyl pyruvate, isoamyl pyruvate, and propyl pyruvate.

27. The method of claim 21, wherein the collection solution comprises
dibasic sodium phosphate;
monobasic potassium phosphate;
sodium pyruvate;
polysorbate 80; and
lecithin derived from sunflower.

28. The method of claim 21, wherein the collection solution does not comprise nutrients for growth of microorganisms.

29. The method of claim 21, wherein the microorganisms are selected from the group consisting of Escherichia coli, Staphylococcus aureus, Salmonella, and Listeria.

30. The method of claim 21, wherein polysorbate 80 is present at a concentration from about 5 grams/liter to about 20 grams/liter, and lecithin is present at a concentration from about 5 grams/liter to about 10 grams/liter.

31. The method of claim 21, wherein the pH buffering system results in a pH in the range of 6 to 8 when the collection solution is mixed with a 1:400 dilution of an acid sanitizer comprising 4.4% peroxyacetic acid and 3.3% octanoic acid; a 1:400 dilution of a high acid sanitizer comprising 13% peracetic acid and 5-20% capric acid.

32. A method of collecting microorganisms from a surface, comprising:
providing a sponge or a swab hydrated with a collection solution comprising pyruvate at a concentration of 10 grams per liter,
contacting a surface with the sponge or swab comprising the collection solution to collect microorganisms on the surface;
optionally storing the sponge or swab comprising the collection solution at a temperature of 2 to 8° C.; and
testing the sponge or swab comprising the collected microorganisms quantitatively using an aliquot of the collection solution in the sponge or swab, or qualitatively by exposing the sponge to a culturing medium.

33. The method of claim 32, wherein the pyruvate is selected from the group consisting of pyruvic acid, sodium pyruvate, ethyl pyruvate, hexenyl pyruvate, isoamyl pyruvate, and propyl pyruvate.

34. The method of claim 32, wherein the microorganisms are selected from the group consisting of Escherichia coli, Staphylococcus aureus, Salmonella, and Listeria.
